(19) United States
(12) Patent Application Publication
(10) Pub. No.: US 2003/0149246 A1
(43) Pub. Date: Aug. 7, 2003

(54) Macromolecular conjugates and processes for preparing the same

(76) Inventor: John C. Russell, Pleasant Prairie, WI (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/062,131

(22) Filed: Feb. 1, 2002

(51) Int. Cl. G01N 33/542; G01N 33/53; C07K 16/46

(52) U.S. Cl. 530/391.1; 435/7.9

(57) ABSTRACT

Making a suspended or soluble macromolecular conjugate comprising binding a first macromolecule to a solid via a stable, disruptable bond, stably linking additional macromolecules, and releasing the macromolecular conjugate, as well as macromolecular conjugates prepared by the method.

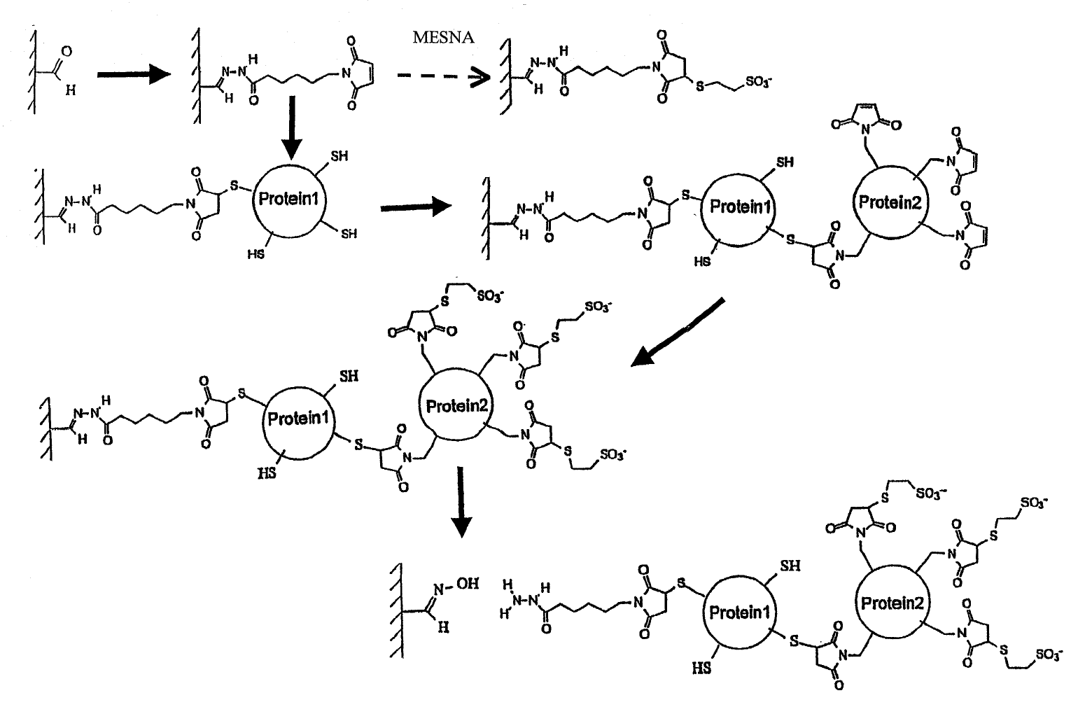

### TECHNICAL FIELD OF THE INVENTION

The present invention pertains to novel macromolecular conjugates, uses of the same, and methods of preparing the same.

### BACKGROUND TO THE INVENTION

The production of complex macromolecular conjugates is important to biotechnological industries. Diagnostic assays and therapeutic agents are two of the multiple technical areas that employ macromolecular conjugates.

In the diagnostic arts, for example, proteins such as antibodies (and antigen-binding polypeptides) are often conjugated to enzymes capable of catalyzing detectable reactions or other macromolecules so that the binding of an antibody to an antigen can be detected. One such antibody-enzyme conjugate illustrative of this technique is a monoclonal FAb fragment conjugated to an alkaline phosphatase. The FAb is capable of binding to a particular analyte and detection of the binding of the antibody with the analyte (and thereby detection of the analyte) is enabled by the conversion by the alkaline phosphatase of a non-luminescent or uncolored substance into a luminescent or colored substance. The skilled artisan, therefore, can use this conjugate to determine the concentration of the analyte in a test sample. Similarly, antigen-binding polypeptides and receptor ligands are frequently conjugated to therapeutic macromolecules in the therapeutic arts. Here, when the conjugate is contacted to an organism (or a tissue or fluid thereof), the antigen-binding polypeptide or macromolecular receptor ligand targets the antigen or receptor to a cell, tissue, or region of the organism. The therapeutic macromolecule, such as a toxin or hormone, is therefore concentrated near the antigen or receptor, preferably increasing the therapeutic index of the therapeutic macromolecule. The skilled artisan will appreciate that many other applications and embodiments of macromolecular conjugates are known and used in the biomedical and other arts.

The preparation of these macromolecular conjugates is typically accomplished by forming reactive moieties on both macromolecules to be conjugated and contacting these macromolecules together under suitable conditions so that a stable bond is formed between these two macromolecules. When one or both of the macromolecules has only one reactive moiety on its surface, the conjugation reaction can be well controlled. Under these circumstances, only one macromolecule of a first type (containing one or more reactive moieties) and only a limited number of macromolecules of a second type (containing only one reactive moiety) can be incorporated into a conjugate.

When each of two (or more) types of macromolecules in a conjugation reaction contain multiple reactive moieties, or when a self-reactive macromolecule comprises multiple sites of reactivity, however, the possibility of uncontrolled network formation during conjugation arises. These conjugates or networks can be large, of variable size, and can incorporate variable numbers of each reactant in the conjugate product. For example, in the FAb-alkaline phosphatase example given above, one FAb might bind three alkaline phosphatases, each of which could bind one to three FAbs, and so on. This typically leads to a population of conjugates with diverse sizes, variable analyte or target binding capacity, and variable reporter, effector, or other end-point modulator content. To reduce this variability, it is desirable to employ one or more methods for controlling the conjugation reaction.

Poorly controlled conjugation can be undesirable for a number of reasons.

In the diagnostic arts, for example, conjugates that are too small (e.g., simple dimers) and other conjugates that are too large, (e.g., conjugates comprising tens of each monomers), may not function well or at all in any one particular application. Similarly, conjugates comprising too large a ratio of one macromolecule to another may lead to decreased sensitivity (e.g., caused by one conjugate binding more than one analyte and yet generating only one unit of signal) or decreased specificity (e.g., caused by an increase in non-specific binding of an analyte binding member to reaction vessels or contaminating substances) or both, and may unnecessarily increase the cost of preparing and using the conjugate (e.g., by incorporating many times the optimum number of an expensive macromolecular precursor into conjugates). Additionally, stability (shelf-life), precision, lot to lot reproducibility, and the like may vary significantly with the average size and distribution of sizes of conjugates.

In the therapeutic arts, for example, an affinity or targeting moiety may be conjugated to an effector molecule. For example, a humanized monoclonal antibody specific for a tumor cell may be conjugated to a toxin. Failure to adequately control the conjugation process can lead to variability in stability of the conjugate, biological half-life (including without limitation degradation and half-life), and therapeutic index (i.e., the therapeutic efficacy at the highest medically-acceptable concentration).

Moreover, the composition of the surface of a conjugate in uncontrolled conjugation process can be variable or difficult to control. Accordingly, undesired aggregation and surface adhesion dictated by surface affinity for other molecules, charge or hydrophobicity, and/or particle diameters can occur.

To control the conjugation process, previous methods have focused on control of pH, temperature, degree of precursor activation, absolute concentration of macromolecular precursors, stoichiometry of precursors, and vigor of mixing of reactants. These methods of controlling the conjugation process have been sufficiently effective to provide industrially useful applications. Additionally, these methods can be usefully combined with the novel aspects of the present invention.

Similarly, populations of conjugates having a wide variety of sizes and compositions can be fractionated by size exclusion chromatography or similar methods. However, this can result in the loss of a large amount of conjugate that is outside the desired size range, and may not be very practical for large-scale applications. Similarly, selection of conjugates by gel chromatography is limited by the relatively low resolution achievable with large polydisperse conjugates. As with prior art methods of conjugation, some prior art methods of conjugate enrichment or purification can be used in conjunction with the novel aspects of the present invention.

Additionally, it is often desirable to have better defined conjugate compositions to aid in quality-assurance.

Therefore, a need exists (in the diagnostic arts, the therapeutic arts, the agricultural and food products processing arts, the chemical production and processing arts, and other arts) to improve macromolecular conjugates by improving control over the process. The present invention addresses this need.

### BRIEF SUMMARY OF THE INVENTION

The present invention provides an improved method for conjugating macromolecules, as well as novel conjugated macromolecules (i.e., conjugates) produced by the method. The present invention also provides a method of detecting analytes comprising contacting the analyte with a macromolecular conjugate obtained through the improved method of producing a diagnostic macromolecular conjugate, a method of providing therapy to an organism in need of therapy comprising an improved method of producing a therapeutic conjugate, and kits comprising novel macromolecular conjugates.

The present inventive method comprises contacting a First Macromolecule to a reactive support to form a solid-bound macromolecular complex. If necessary, either or both steps of activating the First Macromolecule and deactivating the unreacted reactive moieties on the reactive surface are performed. A Second Macromolecule is activated, if necessary, and contacted to the First Macromolecule. After the solid, First Macromolecule and Second Macromolecule bind to form a ternary complex, the bond between the solid and the First Macromolecule is disrupted to provide a macromolecular conjugate that is preferably soluble or dispersable in aqueous solution.

One or more additional optional steps can be performed to add additional macromolecules and smaller molecules or atoms to the Macromolecular Conjugate. Any additional optional steps are preferably performed prior to disruption of the bond between the solid and the First Macromolecule.

The conjugation process is preferably completed by disrupting the bond between the reactive surface and the First Macromolecule to form a suspended, dispersed, or soluble macromolecular conjugate with a technique or reagent that does not substantially diminish the quality of the conjugate, nor substantially diminish the characteristics for which the reactant was added to the conjugate.

Each step of the process, in fact the entire process, is preferably performed under aqueous conditions suitable to maintain the biological activity of an enzyme (e.g., bovine alkaline phosphatase).

Also provided is a macromolecular conjugate with improved characteristics, which may include improving, without limitation, homogeneity of conjugate size, homogeneity of conjugate composition, hydrophilicity or hydrophobicity, surface charge, and spatial arrangement of the macromolecules and other groups incorporated into the conjugate.

The present invention also provides a method of detecting a target or analyte comprising contacting a test sample with the Macromolecular Conjugate of the present invention under conditions suitable to form a complex between the target or analyte and the Macromolecular Conjugate, and detecting the presence or quantity of Macromolecular Conjugate bound to the target or analyte.

The present invention also provides a method of treating an organism, preferably an animal, in need of treatment comprising contacting the organism with a therapeutically effective amount of a Macromolecular Conjugate of the present invention, wherein at least one macromolecule or other chemical group of the Macromolecular Conjugate interacts with a cell, tissue, or molecular component (e.g., a neurotransmitter) of the organism to improve the condition or state of the organism.

### BRIEF DESCRIPTION OF THE DRAWING

The drawing depicts aspects of an embodiment of the present invention described in the Example and is further described within that Example.

### DETAILED DESCRIPTION OF THE INVENTION

Prior art methods of forming macromolecular conjugates provide only limited control over the conjugation process. Accordingly, prior art macromolecular conjugates usually have low uniformity, random and uncontrolled spatial relationships as between the monomers they comprise, and are subject to uncontrolled combination by cross-linking reactions. The present invention provides, among other things, a greater degree of control over conjugation processes. The present invention also improves processes employing the improved method of conjugation as well as the products (i.e., conjugates) produced by the present invention. The present inventive method is preferably performed under aqueous conditions, and more preferably performed entirely under aqueous conditions. The aqueous conditions are even more preferably selected so as to maintain the desired activity of the macromolecules that are conjugated. In an embodiment of the inventive method, the aqueous conditions are selected so as to maintain the catalytic activity of bovine intestinal alkaline phosphatase.

The present inventive method of conjugating a macromolecule comprises linking a First Macromolecule to a solid to form a complex between the solid and First Macromolecule, which frequently is, but need not be, the first step in the present inventive conjugation process. The First Macromolecule is also reacted with a Second Macromolecule, which can be the same or different as the First Macromolecule. The linked Solid-First Macromolecule-Second Macromolecule is optionally then reacted with a Third Macromolecule, and optionally a Fourth Macromolecule. The solid preferably reacts only with the First Macromolecule, and when used, capping compounds. The First Macromolecule preferably does not further react with the solid once any reaction with the Second Macromolecule has occurred. The Second Macromolecule preferably does not further react with the First Macromolecule after reacting with a Third Macromolecule. Similarly, it is preferred that all macromolecular reactions are sequentially performed such that any added macromolecule reacts with only one bound complex, i.e., does not form a bridge between two or more bound complexes, and such that any one macromolecule reacts with only one other type of macromolecule (e.g., a Third Macromolecule) at any one time, and such that any reaction between two macromolecules can occur only once. In theory, there is no limit to the number of macromolecules that can be joined to the conjugate.

The linking of the First Macromolecule to the solid provides a center of synthesis around which further additions to the conjugate can take place. Advantageously, these centers of synthesis are fixed, and usually dispersed, so that cross-linking between the centers of synthesis are either controlled, or preferably avoided. Therefore, the molar quantity of Molecular Conjugates prepared when there is no cross-linking (or dimerization) of conjugates on the solid is preferably equal to, or substantially equal to, the quantity of the First Macromolecule linked to the solid. Substantially all of the First Macromolecule added to the reaction with the solid is preferably converted to the solid-bound state during the present inventive method of making a Macromolecular Conjugate.

Any suitable solid can be used in the inventive method. However, the solid is preferably convoluted, and more preferably porous. While not desiring to be bound by any particular theory, convoluted and especially porous solids are preferred because (i) the convolutions in the surface, or the pores of the solid, tend to shield a macromolecular conjugate growing from one center of synthesis from other macromolecular conjugates growing from other centers of synthesis, and (ii) the large surface areas of convoluted and porous solids provide ample area to allow separation (i.e., distance) between sites of First Macromolecule linkage to the solid, thereby allowing control over the degree of aggregation. The solid is preferably a size-exclusion based chromatography bead or particle. The solid can more preferably comprise, or consist essentially of, cross-linked polyacrylamide, and more preferably agarose.

The First Macromolecule can be reacted with the solid under dilute conditions, and/or with slow reaction kinetics so that the centers of synthesis formed by the reaction of the First Macromolecule with the solid are physically separated. For example, the pH, temperature, and concentration of the First Macromolecule (e.g., an antibody) can be controlled so that the First Macromolecule has a good opportunity to penetrate into the pores of a suitable solid before being bound. Selecting optimal reaction conditions can prevent the majority of the First Macromolecule from binding on the outer surfaces of the particles of solid (i.e., binding of the First Macromolecule to those surfaces of the solid that contact other particles of solid or the walls of the container holding the solid).

Optionally, the Macromolecular Conjugate can be fractionated or purified, for example, using affinity chromatography or size-based selection. The fractionation or purification of the Macromolecular Conjugate can be performed by any suitable technique or combination of techniques. Advantageously, the present inventive Macromolecular Conjugates frequently can be employed in diagnostic applications, therapeutic applications, or other applications without any purification or selection.

If necessary or desired, the solid is treated or prepared to become reactive with the First Macromolecule. When necessary, any suitable moiety or molecule can be used to make the solid reactive with the First Macromolecule. This readily can be achieved by a number of methods including, without limitation, contacting the solid with an activating agent to produce on the surface of the solid a reactive moiety such as, without limitation, a(n) hydroxyl, aldehyde, carboxylic acid, diene, amine, sulfhydryl, phosphoryl, or other reactive chemical moiety on the surface of the solid. Optionally, the reactive moiety can be complex comprising 6 or more atoms other than hydrogen. Biotin, which forms highly stable complexes with avidin, streptavidin and similar molecules, is one of a multiplicity of suitable complex reactive moieties that can be used to activate the surface of the solid.

Advantageously, linkers, including bifunctional linkers, heterobifunctional linkers, and polyfunctional linkers, can be employed in the context of the present invention. Some of the uses and the composition of linkers are understood in the art. Bifunctional linkers preferably have the formula X-R-Y, wherein X is a First Reactive Moiety, R is a spacer, and Y is a Second Reactive Moiety. X and Y, which may be the same (i.e., a homobifunctional linker) or different (i.e., a heterobifunctional linker) and can be any suitable reactive moiety. Suitable reactive moieties include, but are not limited to, a(n) aldehyde, amine, carboxylic acid (activated or in the presence of an activator, e.g., EDAC), diene, hydrazide, hydroxyl, maleimide, NHS ester, phosphoryl, sulfhydryl, thiol, and other reactive chemical moieties. Either X or Y is preferably an aldehyde, a carboxylic acid, a hydrazide, a maleimide, or a thiol. The spacer R can be any suitable substituted or unsubstituted aliphatic or aromatic organic moiety. Suitable organic moieties can, but need not be selected from the group consisting of: methylyne radical, alkyl, cycloalkyl, cycloalkylalkyl, aralkyl, aryl, alkoxyalkyl, haloaryl, hydroxyalkyl, carboxy, carboxyalkyl, alkanoyl, alkenyl, and alkynyl. The spacer moiety advantageously allows control of inter-macromolecular steric restrictions. For this and other reasons, the spacer preferably comprises from 1 to 90 carbon atoms, more preferably comprises 1 to 30 carbon atoms, and yet more preferably comprises 3 to 20 carbon atoms.

The solid, First Macromolecule, Second Macromolecule, and any other macromolecules to be joined to the Macromolecular Conjugate optionally can be reacted with a bifunctional linker or a polyfunctional linker, and preferably a heterobifunctional linker having at least two reactive moieties that can be differentially reacted or activated and reacted. Another option is to treat one or more macromolecules to be joined to the conjugate with reagents that expose a previously hidden or unavailable active group, such as, without limitation, contacting a protein or other macromolecule with dithiothreitol (DTT) to expose sulfhydryl moieties, which are suitably reactive.

Bifunctional linkers allow the facile conjugation of macromolecules and when the bifunctional linker is heterobifunctional, the asymmetry of the linker allows another degree of control over the conjugation reaction. Suitable bifunctional linkers are commercially available from a wide variety of sources. See, e.g., the 2001 Pierce Catalog (Pierce Inc., Rockford, Ill.). Similarly, polyfunctional linkers can be readily synthesized by the skilled artisan. Suitable bifunctional linkers in the context of the present invention include, but are not limited to, ethylene glycol bis[succininimidylsuccinate], NHS ester, N-ε-maleimidocaproic acid, N-[ε-maleimidocaproic acid]hydrazide, N-succinimidyl S-acetylthioacetate, and N-succinimidyl S-acetylthiopropionate. Preferred bifunctional linkers include, but are not limited to, N-Succinimidyl S-Acetylthiopropionate, N-Succinimidyl S-Acetylthioacetate, 2-Iminothiolane (Trauts reagent), 4-Succinimidyloxycarbonyl-Methyl-(2-Pyridyldithio)-Toluene Sulfosuccinimidyl, 4-[N-maleimidomethyl]-cyclohexane-1-carboxylate, N-[gamma-Maleimidobutyryloxy]sulfosuccinimide ester, N-(K-Maleimidoundecanoyloxy) Sulfosuccinimide Ester, Maleimidoacetic Acid N-Hydroxysuccinimide Ester, N-(Epsilon-Maleimidocaproic Acid) Hydrazide, N-(K-Maleimidoundecanoic Acid) Hydrazide, N-(Beta-Maleimidopropionic Acid) Hydrazide, and 3-(2-Pyridyldithio)Propionyl Hydrazide.

Polyfunctional linkers are bifunctional linkers with at least one additional reactive moiety. Preferably, polyfunctional linkers comprise at least three types of reactive moieties, wherein each reactive moiety can be selectively reacted such that the linker can be used to non-randomly and sequentially complex three macromolecules. Alternatively, and also preferred, are polyfunctional linkers comprising only one reactive moiety of a first type, and multiple reactive moieties of a second type. The skilled artisan will appreciate, however, that other polyfunctional linkers are suitable in the context of the present invention.

In certain embodiments, the solid is derivatized with a specific binding pair. A specific binding pair is a pair of specific binding elements that specifically bind together when contacted to each other under suitable conditions. Specific binding pairs allow the formation of a linkage, preferably of predictable character, between the First Macromolecule and a Second Macromolecule. Any suitable specific binding pair can be used in the context of the present invention. As an example without limitation, biotin and avidin and equivalent molecules of the same which are known in the art (e.g., a biotinylated nucleoside and/or streptavidin) are typical of one class of specific binding pairs. In other embodiments, one or both specific binding pairs can be nucleic acids. As an example without limitation, when the First Macromolecule is a nucleic acid and is bound to the surface of the solid, the Second Macromolecule can optionally be another nucleic acid, a nucleic acid binding polypeptide that binds nucleic acids or binds the specific nucleic acid that is the First Macromolecule bound to the solid. In another embodiment, one specific binding element of the specific binding pair can be a polypeptide (e.g., without limitation, an antigenic epitope, a lectin, or a histidinyl oligopolymer capable of binding with high affinity to another macromolecule. Specific binding elements of specific binding pairs can also be selected from the group consisting of biomolecules, which include, for example, and without limitation carbohydrates, dinucleotides, farnesyl moieties, vitamins, and others, and are characterized by their ability or tendency to form naturally specific pairs in cells or organisms, or to be created by cells or organisms in response to a stimulus such as inoculation. Another preferred specific binding pair is an antigen and antibody or antibody-fragment, wherein the antibody or antibody-fragment has affinity for the antigen and is preferably specific for the antigen.

In more complex embodiments of the inventive Macromolecular Conjugate, the growth of the conjugate can be sterically limited by the space that is occupied by the solid support. For example, when a large number of macromolecules are joined in the Macromolecular Conjugate, a substantially hemispherically-shaped molecule can be produced. Advantageously, the link between the solid and First Macromolecule can include a substantially linear moiety to provide distance between the surface of the solid and the First Macromolecule during synthesis in applications where it is desirable to avoid hemispherically-shaped conjugates. Any suitable linear moiety can be used. The linear moiety is preferably long enough to diminish the solid's steric exclusion of the growing conjugate, but short enough to inhibit cross-reaction of the growing conjugate. The skilled artisan will recognize that the maximum and minimum desirable length of the linear moiety will vary from embodiment to embodiment depending on the porosity of the solid, the density of reactive sites on the solid, and the initial quantity of the First Macromolecule contacted to the solid. In this regard, the linear moiety is preferably, at least 5 nm in length, more preferably greater than 10 nm in length, and optionally greater than 25 nm in length. Additionally, the linear moiety is preferably not longer than 700 nm in length, more preferably not longer than 350 nm in length, and yet more preferably not more than 200 nm in length.

By way of example, without limitation, the linker can comprise a polyethylene glycol activated at both ends with —SH groups. The —SH moieties would react with the EMCH-activated support, giving a thiol group at the end of a linear moiety. Polyethylene glycol with a molecular weight of about 2 kDa is known to have a linear length of about 16 nm whereas polyethylene glycol with a molecular weight of about 10 kDa has a linear length of about 80 nm. In contrast, the use of an EMCH linker alone provides only about 1.2 nm of separation between the solid and the First Macromolecule. Advantageously, polyethylene glycol polymers functionalized at both ends are commercially available with a large number of different functional groups including those of interest in the process of the invention. Similarly, other linear moieties, including but not limited to polymers, can be readily functionalized at both ends by the skilled artisan.

Irrespective of whether a bifunctional linker or an active site on the reactants (i.e., solid, First Macromolecule, Second Macromolecule, and/or any other conjugated Macromolecule) is employed to conjugate a reactant molecule in the making of the present inventive Molecular Conjugate, residual reactive moieties on each of these reactant molecules can be inactivated, or converted to other desired functions, after reaction with the solid or other reactant. It is particularly preferred that reactive moieties on the solid remaining after the reaction with the First Macromolecule be inactivated prior to performing additional steps, especially when the reactive moiety on the First Macromolecule will be used to join additional macromolecules to the growing conjugate. For example (without limitation), the First Macromolecule can be activated by causing it to react with a heterobifunctional linker. The First Reactive Moiety of the heterobifunctional linker is covalently bound to the First Macromolecule. The derivatized First Macromolecule and solid are contacted together to form a First Macromolecule:solid complex. Then, a Second Macromolecule (which may be the same or different from the First Macromolecule) and which is reactive with the Second Reactive Moiety of the heterobifunctional linker is contacted to the derivatized First Macromolecule and incubated for a suitable and desirable period of time such that a solid-First Macromolecule-Second Macromolecule stable complex is formed. General chemistry principles suggest, however, that some fraction of the Second Reactive Moieties of the heterobifunctional linker usually will not have reacted with the Second Macromolecule. This usual failure to completely react the Second Reactive Moiety of the heterobifunctional linker frequently (but not always) can be avoided by driving the reaction to completion, but this is most often not necessary and can be undesirable because it requires higher levels of the Second Macromolecule, thereby increasing costs for reactants and disposal of waste products, and because it can increase reaction times to commercially unattractive lengths of time. Accordingly, the Second Reactive Moiety of the heterobifunctional linker can be incompletely reacted.

The existence of residual (i.e., unreacted) Second Reactive Moieties of bifunctional linkers can cause complications before, and more often after, the disruption of the bond holding the conjugate to the surface. First, the unreacted reactive moieties may later react with a Second Macromolecule on another molecule in a population of the Molecular Conjugate. If this were to happen, it would tend to allow uncontrolled cross-reaction of Molecular Conjugates, which as noted in the Introduction can be undesirable. Second, the reactive moieties may be employed in the addition of a Third Macromolecule, a Fourth Macromolecule, or another macromolecule. In this case, the residual unreacted Second Reactive Moiety of the bifunctional linker could compete in the later reaction. Thus, one would need to avoid repetitively using an individual reactive moiety, or accept a certain degree of loss of control of the conjugation reaction.

A third aspect of the unreacted moieties, however, provides an opportunity to usefully improve the inventive Macromolecular Conjugate of the present invention. These unreacted moieties can be inactivated with a Capping Compound, which optionally can be used to usefully modify the characteristic of the produced Macromolecular Conjugate. A Capping Compound is a compound that can be used to inactivate all or substantially all of the unreacted reactive residues of macromolecule or macromolecule-bound linker. Typically Capping Compounds are contacted to the solid-Macromolecular complex in sufficient quantity and for a sufficient period so as to substantially eliminate residual unreacted reactive moieties of a particular type. More than one Capping Compound can be used, however, and these Capping Compounds may be introduced simultaneously or sequentially. Suitable classes of Capping Compounds include, but are not limited to, detectable compounds, charge altering compounds, polymeric compounds, steric spacers, and specific binding elements of a specific binding pair. By way of example, without limitation, haloacetamides and maleimides can be suitably used to cap sulfhydryl reactive moieties, whereas thiol-containing reagents can be suitably used to cap maleimide reactive moieties.

The capping compound can be disposed to the surface of the conjugate by introducing it after addition of the final layer of macromolecule. Use of an appropriate capping compound can impart advantageous properties to the Macromolecular Conjugate. For example, the capping compound can decrease nonspecific binding of a conjugate used for immunodiagnostics, and can protect the conjugate from destruction by a patient's immune system.

The group of suitable detectable Capping Compounds comprises (without limitation) small organic fluorescent dyes such as fluorescein-5-maleimide. The use of these capping compounds allows the conjugate to be detected, traced, and quantified by fluorescent detection systems. When used to cap linkers disposed to the core (rather than the surface) of a Molecular Conjugate, these fluorophores do not increase the tendency of the Molecular Conjugate to stick to other molecules and surfaces.

The group of suitable charge altering Capping Compounds comprises (without limitation) alkanyl, alkenyl, alkynyl compounds having 2 to 20 carbon atoms and one or more charged moieties. Suitable charged moieties include, but are not limited to, amino, carboxyl, sulfhydryl, phosphoryl, and thiophosphoryl moieties. Zwitterionic charged moieties (such as certain amino acids) can be preferred for their ability to increase solubility in aqueous solutions and other reasons. Suitable examples of charge altering Capping Compounds include, but are not limited to, compounds comprising cystamine, thioacetic acid, cysteine (2-amino-3-mercaptopropionic acid), imidazole, aspartic acid (2-aminobutanedioic acid), and lysine (2, 6-diaminohazanoic acid) moieties. The sulfhydryl moiety of the cysteine, for example, can react with suitable unreacted Second Reactive Moieties, thereby leaving an amino moiety and a carboxylic acid moiety, which tend to be positively and negatively charged (respectively) at pH from about 3 to about 10.

The group of suitable steric spacing Capping Compounds comprise, but are not limited to, derivatives of polyethylene glycol, and polysaccharides.

The group of suitable polymeric Capping Compounds comprises, but is not limited to, dextran, polyethylene glycol, and polysaccharides. Among preferred polysaccharides are heparin and sialic acids, both of which are known to provide beneficial effects on compounds introduced into animals, including humans.

Other capping compounds useful in the context of the present invention include thioacetic acid, N-ethylmaleimide, iodoacetic acid, and C₁-C₂₅ haloaliphatic compounds.

The synthesis of the Macromolecular Conjugate optionally also includes the step of disrupting the bond between the solid and the First Macromolecule so that the Macromolecular conjugate is released from the solid. The Macromolecular Conjugate can be insoluble, but is preferably soluble or dispersable in aqueous solutions. When the Macromolecular Conjugate is not soluble, but is dispersable in aqueous solution it is more preferably fully dispersed.

Optionally, Capping Compounds useful in rendering reactive groups of the solid unreactive with the First Macromolecule can also be introduced before the First Macromolecule, with the First Macromolecule, or after the First Macromolecule is contacted to the solid. Advantageously, Capping Compounds that reduce the reactivity of the solid can be used to reduce bridging (i.e., cross-reaction) of growing Macromolecular Conjugates by competing for solid surface binding sites and keeping the sites of attachment for the First Macromolecule sparse. Similarly, Solid-specific Capping Compounds can be used to terminate the reaction of the First Macromolecule with the solid after a suitable degree or period of reaction has occurred.

A capping compound can also be used to provide a reactive group different from the one which is capped. For example, EMCH, which caps sulfhydryl groups can be selectively modified after release of the Macromolecular Conjugate from the solid.

Any suitable solid can be used in the context of the present invention. The solid surface can, for example, be selected from the group consisting of glass, paper, agarose, polyacrylimide, polydextran, polyvinylpyrolidone, and polystyrene. Discrete particles of the solid are preferably visible to a normal, healthy human eye under 20× magnification, and more preferably visible to the unaided human eye. Particles of the solid preferably comprise a convoluted or dimpled surface so that centers of synthesis are separated from each other. Examples of solids with convoluted surfaces suitable in the context of the present invention include, without limitation, nucleopore substrates, and lithographic surfaces commonly employed in the microelectronic arts, and sometimes employed in the biological arts Examples of porous solids suitable in the context of the present invention include, without limitation, gel filtration particles such as Sephadex G-25 and Sepharose CL2B. Porous solids useful in the context of the present invention preferably have average pores sufficiently large to admit globular molecules having a molecular weight of 50,000 daltons, more preferably having a molecular weight of 200,000 daltons, and even more preferably, having a molecular weight of 500,000 daltons or more. Optionally, the porous solids useful in the context of the present invention preferably have average pores sufficiently large to admit globular molecules having a molecular weight of 10,000,000 daltons, which porous solids are preferred for the preparation of a Macromolecular Conjugate having a higher number of macromolecule layers.

The bond between the First Macromolecule and the solid can be of any suitable form. For example, the bond can be a stable bond, for example, of covalent, ionic, or hydrophobic character. Stable bonds are most useful when the solid is to be incorporated into the Macromolecular Conjugate. When the bond is a stable non-covalent bond, the bond preferably has a Kd of less than 10⁻⁵ M, and more preferably less than 10⁻⁸ M, and yet more preferably less than 10⁻¹⁰ M, in an aqueous buffer at pH 7.0 that is isotonic with human serum.

Preferably, however, the bond between the First Macromolecule and the solid is disruptable. A disruptable bond is one which can be severed under predictable conditions, which do not destroy or disaggregate the Molecular Conjugate, and preferably do not inactivate bovine intestinal alkaline phosphatase. Disruptable covalent bonds suitable in the context of the present invention include, but are not limited to, hydrazone, semicarbazone, Schiff Base, disulfide, vicinal diol, ester. Suitable disruptable non-covalent bonds include, but are not limited to ionic, antibody-antigen, hydrogen bond (e.g. via complementary nucleic acid sequences).

The First Macromolecule, Second Macromolecule, and any other included Macromolecules (collectively Macromolecules) are independently selected from among all macromolecules of interest. For example, these Macromolecules can be proteins, nucleic acids, polymers, saccharides, and/or combinations of these classes of compounds. When a First Macromolecule or Second Macromolecule is a protein or a nucleic acid it is preferably reactive at at least two positions (i.e., bifunctional), more preferably reactive at at least three positions, yet more preferably reactive at at least four positions, and optionally at more than 10, or more than 20 positions.

In theory, there is no upper limit to the number of reactive sites on a Macromolecule incorporated into the Macromolecular Conjugate of the present invention. Nonetheless, in most embodiments of the present invention the macromolecules incorporated into the Macromolecular Conjugate will have no more than about 200 reactive sites per molecule, and optionally, no more than about 50 reactive sites per molecule. In some embodiments, the First Macromolecule, or Second Macromolecule, or other Macromolecule joined to the present inventive Macromolecular Conjugate is an end-point molecule.

An end-point molecule can be a macromolecule capable of generating or attenuating a detectable signal. End-point molecules can also function to bind other molecules or surfaces, catalyze reactions, exert a toxic effect, or exert a beneficial or therapeutic effect. End-point molecules suitable in the context of the present invention include, but are not limited to, enzymes (especially enzymes known in the art to convert a colorless reactant to a colored reactant, or to generate light when contacted with a substrate under suitable conditions), fluorophores (including, but not limited to fluorescent dyes and fluorescent proteins), radio-labeled proteins, nucleic acids, and other molecules, co-factors for enzymes, luminescent molecules, and chromophores. The interactions that can be usefully initiated by end-point molecules include appropriately specific and selective interactions productive of groups or complexes which are themselves readily detectable, for example, by calorimetric, spectrophotometric, fluorometric, or radioactive detection procedures. Such interactions can take the form of protein-ligand, enzyme-substrate, antibody-antigen, carbohydrate-lectin, protein-cofactor, protein-effector, nucleic acid-nucleic acid and nucleic acid-ligand interactions. Additional examples of such ligand-ligand interactions include, but are not limited to, dinitrophenyl-dinitrophenyl antibody, biotin-avidin, oligonucleotide-complementary oligonucleotide, DNA-DNA, RNA-DNA and NADH-dehydrogenase. Either one of each of such ligand pairs can be an end-point molecule.

In one embodiment, the First Macromolecule of the soluble or suspended Molecular Conjugate is a protein. The protein preferably has at least at least 3 reactive sites, and preferably has at least five reactive sites. Additionally, the protein preferably has a molecular weight of at least 2,000 daltons, more preferably of at least 10,000 daltons, and yet more preferably of at least 30,000 daltons. In this embodiment, the protein is contacted to the solid to form a surface-bound protein complex, and preferably is a disruptable covalent bond. Preferably, at least a majority of the protein binds to surfaces within indentations, convolutions, or pores of the solid. The unreacted reactive sites on the solid preferably are inactivated after binding with the First Macromolecule. If necessary, the protein is activated to make it reactive with the Second Macromolecule or an activated form of the Second Macromolecule. Similarly, the Second Macromolecule is activated if it is necessary to make it reactive with the protein or the activated protein. The Second Macromolecule is then contacted to the bound First Macromolecule. The Molecular Conjugate optionally can then be further treated as described above. Such further treatment can be performed prior to, or after, disruption of the stable bond between the solid and the residue of the First Macromolecule, which is incorporated into the Molecular Conjugate.

In an alternative embodiment, the present invention provides a macromolecular conjugate and a method for preparing a suspended or soluble macromolecular conjugate. The First Macromolecule can be any suitable macromolecule having a plurality of reactive moieties capable of interacting (directly or through an activated intermediate) with a Second Macromolecule that has a plurality of reactive moieties that are capable of reacting with the First Macromolecule (in the form employed in the present inventive process whether activated or naturally reactive). The First Macromolecule can be an antibody, or another protein, or another suitable macromolecule. The method comprises the steps of providing a reactive surface, and a First Macromolecule that are capable of reacting together when brought into contact under suitable conditions to form a stable, disruptable bond to form a surface-bound macromolecule. If necessary, the First Macromolecule, or the Second Macromolecule, or both are activated to make them mutually-reactive and the First Macromolecule-solid complex and Second Macromolecule are brought into contact to form a solid-surface:First Macromolecule:Second Macromolecule stable complex.

In each case, the production of the soluble or dispersible Macromolecular conjugate includes disrupting the stable bond between the solid-surface and the Macromolecule Conjugate in a liquid medium to yield a suspended or soluble Macromolecule Conjugate.

The present inventive method allows the production of novel and useful conjugates.

For example, the present invention provides, among other things, a composition comprising a population of conjugates, wherein each conjugate of the population comprises a single antibody.

In one embodiment, it is possible to produce a conjugate comprising a definite number of antibodies specific for an analyte, receptor, or other desired target molecule. In some preferred embodiments, all or substantially all of the conjugates in a population of conjugates comprise a single antibody. In other preferred embodiments, each conjugate comprises between 2 and 30 antibodies, and more preferably all or substantially all of the conjugates in a population of conjugates comprise the same number of antibodies. In the context of a diagnostic assay this can be advantageous because every conjugate bound to analyte will bind to only one molecule of analyte. In distinct contrast, prior art aggregated conjugates produced by traditional methods often comprise a multiplicity of antibodies and end-point molecules (e.g., fluorophores or enzymes). Thus, these conjugates bind a variable quantity of analyte, but generate a quantity of signal determined by the quantity of included end-point molecule rather than analyte. Thus, the present inventive conjugate has better accuracy and sensitivity than prior art conjugates.

In another embodiment, a First Macromolecule, which optionally can be an antibody specific for a desired target molecule is conjugated to a Second Macromolecule, which optionally can be a serum albumin, a fluorescent protein, or another macromolecule. When the Second Macromolecule is a fluorescent protein it is preferably selected from the group consisting of R-phycoerythrin, B-phycoerythrin, allophycocyanin, and phycobiliprotein (commercially available, e.g., from Prozyme Inc., San Leandro, Calif.). The Second Macromolecule is in turn conjugated with a Third Macromolecule which serves as a spacer to separate the Second Macromolecule from the Fourth Macromolecule. A suitable spacer-Third Macromolecule preferred in the context of the present invention is serum albumin, however, the skilled artisan will appreciate that many suitable spacer molecules exist. The Third Macromolecule (spacer) is in turn conjugated with a Fourth Macromolecule, which can be the same or different as the Second Macromolecule, and preferably is fluorescent. Optionally, 1 to 10 additional layers of conjugation, preferably 2 to 5 additional layers of conjugation, are present such that a soluble or dispersable conjugate comprising a single First Macromolecule, 1 to 10, and preferably 1 to 4 Second Macromolecules, a layer of Third Macromolecules covering the layer of Second Macromolecules, and a layer of Fourth Macromolecules separated from the layer of Second Macromolecules by the layer of Third Macromolecules, and optionally comprising additional layers of macromolecules is obtained. Optionally, some or all of the macromolecules of the inventive Macromolecular Conjugate are joined by linker molecules as discussed above. Additionally, the layer of Fourth Macromolecule and any additional layers of Macromolecules optionally can consist of a mixture of fluorescent macromolecules and spacer macromolecules characterized in that they do not substantially quench the fluorescence of the adjacent fluorophores, or that they quench in a way that can be controlled in the subsequent use of the conjugate.

Similarly, the present invention provides a Molecular Conjugate comprising a single analyte-specific antibody, a multiplicity of specific binding member molecules, and a multiplicity of end-point molecules.

In another embodiment the Macromolecular Conjugate comprises three layers of macromolecules, wherein at least two of the three layers comprise a plurality of macromolecules, and wherein the layers form a surface in three dimensions.

In yet another embodiment, the conjugate comprises one specific binding element of a specific binding pair, a plurality of end-point molecules, and a plurality of spacer molecules, wherein the spacer molecules substantially separate the end-point molecules.

A population of conjugates, wherein substantially each conjugate of the molecule comprises from 1 to 100 molecules of a specific binding member, each of the specific binding members is disposed on the surface of the conjugate, and substantially each conjugate comprises a core, wherein the core comprises at least one molecule that is not disposed on the surface of the conjugate. This embodiment is advantageous because, inter alia, because the molecules disposed to the core of the conjugate can be inexpensive spacing molecules, have a useful flouroscence, or the like, whereas the molecules disposed to the outer surface of the conjugate can be used to interact with analytes, therapeutic targets, and other entities in contact with the Macromolecular Conjugate.

In yet another embodiment, the present invention provides a Macromolecular Conjugate in which one of the macromolecules, preferably the First Macromolecule, comprises an optically detectable macromolecule. The optically detectable molecule is preferably a chromophore or a fluorophore, and is more preferably a phycobiliprotein such as, without limitation, R-phycoerythrin, B-phycoerythrin, or allophycocyanin. The chromophore renders the Macromolecular Conjugate optically distinguishable from the other macromolecules of the conjugate such that the final conjugate can be quantified according to the optical qualities of the optically detectable macromolecule.

In this embodiment, when the First Macromolecule is optically detectable, the number of “particles” (i.e., the number of conjugates in the population) of the Molecular Conjugate is limited by the number of molecules of the First Macromolecule added to the support. This forms a nucleus around which all other macromolecules are added layer by layer. When the final conjugate is released from the solid it contains one central First Macromolecule.

Advantageously, R-Phycoerythrin can be selected as the First Macromolecule in conjugates comprising alkaline phosphatase, which is an end-point molecule used in many commercially available medical diagnostic and research use assays. R-Phycoerythrin has a very high extinction coefficient at 565 nm, whereas alkaline phosphatase and antibodies do not absorb light at 565 nm. Therefore, the exact molar concentration of a Molecular Conjugate of this embodiment is easily determined regardless of the number of Alkaline Phosphatase and antibody (or antibody-derived) molecules that are included in the conjugate. This can be advantageous in assessing the quality of the conjugate.

The present invention also provides a method of detecting a target or analyte comprising contacting a test sample with the Macromolecular Conjugate of the present invention under conditions suitable to form a complex between the target or analyte and the Macromolecular Conjugate, and detecting the presence or quantity of Macromolecular Conjugate bound to the target or analyte.

The present invention also provides a method of treating an organism, preferably an animal, in need of treatment comprising contacting the organism with a therapeutically effective amount of a Macromolecular Conjugate of the present invention, wherein at least one macromolecule of the Macromolecular Conjugate interacts with a cell, tissue, or molecular component (e.g., a neurotransmitter or cytokine) of the organism to improve the condition or state of the organism.

Suitable macromolecules, which are end-point molecules, for incorporation in the Macromolecular Conjugate of the present invention so as to achieve a therapeutic effect include toxins, autocrines, paracrines, exocrines, radioisotopes, ligands for receptors (including agonists and antagonists of all types), receptor fragments, antibodies, antigen binding polypeptides derived from antibodies or their coding sequence, neuromodulators, antigenic fragments from pathogens, immunosuppresants, and a variety of other macromolecules.

Suitable end-point molecules can also include macromolecules that are disposed to the interior of the conjugate, which comprise small molecules lined to the macromolecule in such a way that when engulfed by and processed by the target cell, these small molecules are released in an active form capable of modifying the behavior of the target cell. For example, a therapeutic molecule can be linked via a hydrazone to a macromolecule of the Macromolecular Conjugate, such that when the Macromolecular Conjugate is internalized by a target cell the linkage between a macromolecule of the Macromolecular Conjugate and a small molecule is disrupted and the small molecule exerts a therapeutic effect on the cell.

The present invention also provides a pharmaceutical composition comprising a pharmaceutically acceptable carrier and a therapeutically effective amount at least one Macromolecular Conjugate of the present invention. Any suitable carrier can be used in the pharmaceutical composition, which will depend in part on the particular means or route of administration, as well as other practical considerations. Such practical considerations include, but need not be limited to, providing a carrier suitable for the solubility of the Macromolecular Conjugate, avoiding chemical reactions with the Macromolecular Conjugate, and protection of the Macromolecular Conjugate from inactivation or degradation prior to delivery to target cells, tissues, and systems.

The pharmaceutically acceptable carriers described herein, for example, vehicles, excipients, adjuvants, or diluents, are well known to those who are skilled in the art and are readily available to the public. Accordingly, there are a wide variety of suitable formulations of the pharmaceutical composition of the present invention. The following formulations are merely exemplary and are not meant to be limiting.

Injectable formulations are among those formulations that are preferred. The requirements for effective pharmaceutical carriers for injectable compositions are well known to those of ordinary skill in the art (See Pharmaceutics and Pharmacy Practice, J. B. Lippincott Company, Philadelphia, Pa., Banker and Chalmers, eds., pages 238-250, (1982); ASHP Handbook on Injectable Drugs, Toissel, 4th ed., pages 622-630 (1986)). Such injectable compositions preferably can be administered intravenously or locally, i.e., at or near the site of a disease, injury, dysfunction, or other condition in need of treatment.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and sterile suspensions that can include suspending agents, solubilizers, thickening agents, stabilizers, and preservatives. The Macromolecular Conjugate may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.0005% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% by weight to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations are well known to those of skill in the art and are suitable in the context of the present invention. Such formulations are typically applied to skin or other body surfaces.

Formulations suitable for oral administration can consist of (a) liquid solutions, such as an effective amount of the Macromolecular Conjugate carried or suspended in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard-shelled or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The Macromolecular Conjugate useful in the present inventive method, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the Macromolecular Conjugate can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition to the above described pharmaceutical compositions, the Macromolecular Conjugate can be formulated as inclusion complexes, such as cyclodextrin inclusion complexes, or in liposomes.

The present invention also provides a kit. The kit includes a reactive conjugate complex, and a cleavage reagent. The reactive conjugate complex comprises a solid bonded with a First Macromolecule, which First Macromolecule in turn is directly or indirectly covalently complexed with a Reactive Macromolecule. The cleavage reagent is capable of cleaving the bond between the solid and the First Macromolecule preferably under conditions that do not inactivate bovine intestinal phosphatase. The kit can also include an activation reagent. The activation reagent can be contacted to a protein of interest to make it reactive with the Reactive Macromolecule. Advantageously, the skilled artisan can then react any protein of interest with the reactive conjugate complex. The resulting product of the skilled artisan's work is a rapidly obtained conjugate that can have a preselected number of protein molecules of interest, and that can be reproducibly prepared.

### EXAMPLES

### Example 1

This Example illustrates the preparation and initial characterization of conjugates comprising of alkaline phosphatase and antibodies specific for Thyroid Stimulating Hormone (TSH).

In this Example, a number of Molecular Conjugates are prepared on a porous solid. Each prepared Molecular Conjugate comprises R-phycoerythrin (RPE) as the First Macromolecule, from 1 to 5 layers of alkaline phosphatase, and a final layer consisting of an antibody specific for the alpha subunit of thyroid stimulating hormone. The size and TSH-specific ELISA activity of the prepared Molecular Conjugates are compared.

The drawing schematically depicts the process of controlled conjugation used in this Example. An agarose support was oxidized with periodate to give immobilized aldehydes. The hydrazide function of the heterobifunctional linker N-[ε-Maleimidocaproic acid]hydrazide (EMCH) was reacted with the aldehydes to give a maleimide group linked to the support via the hydrazone. A sulfhydryl-activated protein was reacted with the maleimide to form a stable thioether linkage, which was still held to the agarose support. The remaining maleimide groups were destroyed by adding the sodium salt of Mercaptoethanesulfonic acid (MESNA). After washing the agarose to remove unbound reactive materials a second protein containing maleimide groups was added. The maleimide groups reacted with remaining —SH groups on the first protein, again forming a thioether linkage. An excess of the second protein was added so that all the available binding locations on the first protein were occupied. Any remaining unreacted sulfhydryl groups on the first protein were available for reaction only with small molecules. The exposed surface of the growing conjugate consisted of the second protein with maleimide groups available for reaction with a third protein, which optionally could have been a repeat of the first protein, containing sulfhydryl groups, but in the present case was either a repeat of the second protein, this time activated with sulfhydryl groups, or the antibody activated with sulfhydryl groups. When all the desired layers of protein were added, the remaining maleimide groups were converted to unreactive thioethers with MESNA. The conjugate was then released from the support by adding hydroxylamine. While not desiring to be bound by any particular theory, it is believed that the hydroxylamine reacts with the hydrazone linkage holding the conjugate to the support, thereby releasing the conjugate as a hydrazide.

To oxidize the agarose, about 20 mL of Sepharose CL2B (from Sigma) slurry was washed with water, suspended in 20 mL of excess water to yield a 50% w/v slurry. To the slurry was added 200 μL of 100 mM NaIO₄ and the mixture was inverted several times to mix. After 75 min at room temperature (about 25° C.), 1 mL of glycerol was added and the mixture was inverted several times to mix. After 15 min, the column was drained in a column fitted with a frit, washed with several column volumes of water and then 1 column volume of phosphate buffered saline (10 mM sodium phosphate, 150 mM sodium chloride, pH 7.2; PBS).

To activate the RPE, 200 μL of R-Phycoerythrin (from Prozyme) at 10 mg/mL in 100 mM triethanolamine HCl at pH 7.6 was added 10 μL of 100 mM N-Succinimidyl S-acetylthioacetate (SATA) in dimethylformamide. After 1 hour at room temperature 10 uL 50% hydroxylamine was added. After 40 minutes at room temperature, the mixture was desalted on a Sephadex G25 column previously equilibrated with a buffer containing 10 mM sodium phosphate, 150 mM sodium chloride, 5 mM EDTA, at pH 7.2, collecting 550 μL. The concentration of activated RPE, which was determined spectrophotometrically by the absorbance at 565 nm, was 10.1 μM, and the number of SH groups per molecule, which was determined using Ellman's reagent, was 22.5.

To activate the alkaline phosphatase, to 500 μL of Calf Intestinal Alkaline Phosphatase at 10 mg/ml (from Boehringer Mannheim) at 100 mM triethanolamine HCl, pH 7.6 was added 50 μL 1M sodium phosphate pH 7.5 and 25 μL 100 mM SATA in dimethylformamide. After 1 hour at room temperature, was added 25 uL 50% hydroxylamine. After 40 minutes at room temperature, the mixture was desalted on Sephadex G25 into a buffer comprising 10 mM sodium phosphate, 150 mM sodium chloride, and 5 mM EDTA at pH 7.2, collecting 1.2 mL. The concentration, which was determined spectrophotometrically by the absorbance at 280 nm, was found to be 30.5 μM and the number of SH groups per molecule, which was determined using Ellman's reagent, was found to be 15.3.

The following procedure was used to make Alkaline Phosphatase activated with maleimido-functional groups. To 500 uL Alkaline Phosphatase (Boehringer Mannheim) at 10 mg/mL in 100 mM triethanolamine HCl, pH 7.6 was added 50 μL IM sodium phosphate at pH 7.5 and 25 μL of 100 mM γ-maleimidobutyric acid N-hydroxysuccinimide ester (GMBS) in dimethylformamide. After 100 minutes at room temperature, the mixture was desalted on Sephadex G25 into a buffer comprising 10 mM sodium phosphate, 150 mM sodium chloride, and 5 mM EDTA at pH 7.2, collecting 1.2 mL. The concentration, which was determined spectrophotometrically by the absorbance at 280 nm, was found to be 29.8 μM, and the number of maleimide groups per molecule, which was determined by the change in absorbance at 300 nm after addition of MESNA to destroy the maleimide chromophore, was found to be 15.4.

The following procedure was used to prepare a maleimide-activated antibody. To 200 uL Anti-TSHₐₗₚₕₐ IgG (from Genzyme) at 7.03 mg/mL in 100 mM sodium phosphate, 150 mM sodium chloride, at pH 7.2 was added 10 μL 100 mM GMBS in dimethylformamide and 2 μL 1M Na₂CO₃ to give pH 7.7. After 100 minutes at room temperature, the mixture was desalted into a buffer comprising 10 mM sodium phosphate, 150 mM sodium chloride, and 5 mM EDTA, at pH 7.2, collecting 550 uL. The concentration, which was determined spectrophotometrically by the absorbance at 280 nm, was found to be 15.9 μM and the number of SH groups per molecule, which was determined by the change in absorbance at 300 nm after addition of MESNA to destroy the maleimide chromophore, was found to be 28.5.

The following procedure was used to prepare a sulfhydryl-activated antibody. To 100 μL Anti-TSHₐₗₚₕₐ IgG (Genzyme) at 7.03 mg/mL in 100 mM sodium phosphate, 150 mM sodium chloride, pH 7.2 was added 5 μL 100 mM SATA in dimethylformamide and 1 μL 1M Na₂CO₃. After 1 hour at room temperature, was added 10 μL of 50% hydroxylamine. After 40 minutes at room temperature, the mixture was desalted into 10 mM sodium phosphate, 150 mM sodium chloride, and 5 mM EDTA at pH 7.2, collecting 550 uL. The concentration, which was determined spectrophotometrically by the absorbance at 280 nm, was found to be 11.7 μM and the number of SH groups per molecule, which was determined using Ellman's reagent, was found to be 25.4.

The following procedure was used to activate oxidized agarose with EMCH. A volume of 6.0 mL of oxidized agarose was poured into a 20 ml column fitted with a frit. The column was washed with 20 mL of a buffer containing 10 mM sodium phosphate, 150 mM sodium chloride, 2 mg/mL CHAPS, and 5 mM EDTA at pH 7.2. (PCE). A stopper was placed on the end of the column, and 3 mL PCE and 90 μL of 100 mM EMCH in dimethylformamide was added and the entire mixture vortexed to disperse the reagent into the resin. After 30 minutes at room temperature, the column was drained, washed with 15 mL cold TC75E buffer (100 mM Tris, 0.2% CHAPS 5 mM EDTA, at pH 7.5) and placed on ice.

To immobilize RPE-SH on activated agarose the following procedure was used. To each of eight 2 mL columns was added 600 μL of the EMCH-treated resin, and the mixtures were washed with 2 mL of TC75E. The columns were stoppered, 200 μL of TC75E added, and the columns were placed on ice. After cooling, 0.200 mole (198 μL) RPE-SH was added to each, and the resins thoroughly dispersed by vortexing. The columns were kept on ice with occasional vortexing. After 20 minutes, 10 μL of 100 mM MESNA was added to each column and the mixtures vortexed and placed on ice. After 5 minutes, the columns were drained and washed with 3.0 mL cold TC8E (100 mM Tris, 0.2% CHAPS, 5 mM EDTA, at pH 8). The absorbance of the first 1.5 mL of the effluent was measured spectrophotometrically at 565 run to determine the quantity of RPE-SH in the effluent. This was subtracted from that originally added to determine the quantity of RPE bound to the support.

The following procedure was used to conjugate Alkaline Phosphatase-Maleimide, Alkaline Phosphatase-SH, and RPE-SH. Except for the time spent draining and washing, the columns were kept in an ice bath throughout the conjugation procedure. Addition steps are shown in Table 1, labeled 1S, 2M, 3S, etc. to indicate which layer of protein is being added (wherein 1 represents the First Macromolecular core of the conjugate and whether the added protein was activated with sulfhydryl (S) or maleimidyl (M) groups). The quantity of activated protein indicated in Table 1 was added to each, along with 100 μL of 1 M magnesium chloride and sufficient TC8E to give at least 250 μL of liquid above the settled resin (the minimum needed for complete dispersal of the resin on vortexing). The columns were incubated in ice for 30 minutes, with vortexing 2 to 3 times during the incubation. They were then drained and the effluent recycled through the column for a total of approximately one column volume. The columns were then washed with 1.5 mL TC8E, collecting the effluent. The columns were stoppered and the next addition of activated protein performed. The absorbance at 280 nm of the effluents was measured to determine the quantity of remaining protein (for alkaline phosphatase, an extinction coefficient at 280 nm of 140000 M⁻¹ cm⁻¹ was used). Prior to reading the absorbance at 280 μm, 10 μL of 100 mM MESNA was added to effluents containing alkaline phosphatase-maleimide to eliminate the maleimide chromophore. From this, the quantity of activated protein bound in each step was calculated.

The following procedure was used to conjugate antibody-maleimide and Antibody-SH. After the final addition and incubation of activated Alkaline Phosphatase according to Table 1, the columns were washed with 1.5 mL TC75E, stoppered and placed in ice bath. After 10 min, 200 μL of cold TC75E and the indicated quantity of activated IgG were added. The mixture was vortexed and placed on ice. After 5 minutes, 100 μL of IM magnesium chloride was added, and the mixture vortexed. After 30 minutes in an ice bath, the columns were drained and washed with 1.5 mL TC8E. The absorbance at 280 nm of the effluents was measured and the quantity of IgG remaining in the effluent calculated using an extinction coefficient at 280 nm of 210000. From this, the quantity of IgG bound to the resin was calculated.

The following procedure was used to release the conjugates from the support.

After washing the unbound IgG from the resins, the columns were stoppered, 200 μL of TC8E and 5 μL of 100 mM N-ethylmaleimide were added to deactivate residual SH groups. The mixtures were vortexed to disperse the resin and the columns were placed on ice. The columns were left on ice until protein addition and washing was complete for all columns. To deactivate residual maleimide groups, 10 μL of 100 mM MESNA was added, the mixtures vortexed, and the columns returned to ice. After 10 minutes, 20 μL of 50% hydroxylamine was added, the columns vortexed to disperse the resin, and incubated for 60 minutes at room temperature. The columns were then drained directly into PD10 desalting columns (from Pharmacia), which were pre-equilibrated with PBS, and the products were washed through with TC8E and PBS, collecting 2.5 mL of product. Concentrations of product were determined based on the absorbance at 565 nm, and yields calculated based on recovery of RPE color. Protein concentrations of the conjugates were measured by the BCA assay (reagents from Pierce) using the enhanced protocol according to the manufacturers instructions.

The samples were analyzed by HPLC. To 300 μL of each conjugate was added 30 μL of 10 mg/mL CHAPS in PBS. A sample of 20 μL was passed through a Whatman Macrosphere GPC 1000A 250×4.6 mm column with 1 mg/mL CHAPS in PBS as the mobile phase at 0.2 mL/min, monitoring at 280 nm and 566 nm with a diode array detector.

The phosphatase activities of the prepared conjugates were compared. Dilutions in a buffer containing 50 mM bis-tris-propane, 150 mM NaCl, 10 mM MgCl₂, 1 mM ZnCl₂ pH 7.2 and 10% of a commercially-available non-specific binding blocking reagent (i.e., Superblock™ (Pierce)) (Pierce) were prepared of each conjugate to give 10 nM bound Alkaline Phosphatase, based on the absorption of activated Alkaline Phosphatase in the conjugation reactions and assuming recovery of Alkaline Phosphatase was proportional to recovery of R-Phycoerythrin color. To 5.0 μL of each conjugate in the wells of a microplate was added 100 μL of substrate (PNPP, Pierce), the mixtures agitated and the absorptions at 405 nm measured over 5 minutes. Unconjugated alkaline phosphatase at 10 nM was used as a reference.

The conjugates were compared in TSH ELISA assays. The wells of a 96-well microtiter plate were coated with a monoclonal antibody specific for the beta subunit of TSH (20 μg/mL in PBS for 60 min at 37 degrees and blocked with a commercially-available non-specific binding blocking reagent (i.e., Superblock™ (Pierce)). To the wells was added 25 μL of standard solutions containing TSH, which were are used as calibrators for a commercial TSH assay. The plate was covered to prevent evaporative loss of sample volume and incubated for 3 hours at 37° C. The wells were drained, washed five times with water and 100 μL of conjugate in a buffer containing 50 mM bis-tris-propane, 150 mM NaCl, 10 mM MgCl2, 1 mM ZnCl₂, and 10% A commercially-available non-specific binding blocking reagent (i.e., Superblock™ (Pierce)), at pH 7.2 was added. The conjugate concentration was 40 pM RPE for one test and 200 ng/mL protein for another test. After 3 hours at 37° C., the wells were drained and washed fives times with water. One hundred μL of substrate was added and the plate placed at 37° C. The absorbance at 405 nm was measured at 30-second intervals over 30 minutes. The Vₘₐₓ was calculated for each well.

TABLE 1
This table identifies the composition of the macromolecules used to prepare the
various conjugates prepared in Example 1.

| | A | B | C | D | E | F | G | H |
|---|---|---|---|---|---|---|---|---|
| 1S:nmole RS bound | 0.176 | 0.172 | 0.173 | 0.168 | 0.174 | 0.178 | 0.171 | 0.176 |
| 2M:nmole APM added | 0.800 | 0.800 | 0.800 | 0.800 | 0.800 | 0.800 | 0.800 | 0.800 |
| 2M:nmole AOM bound | 0.609 | 0.594 | 0.624 | 0.633 | 0.633 | 0.635 | 0.635 | 0.636 |
| AP/RPE | 3.5 | 3.5 | 3.6 | 3.8 | 3.6 | 3.6 | 3.7 | 3.6 |
| 3S:nmole APS added | | 1.80 | 1.80 | 1.80 | 1.80 | 1.80 | 1.80 | 1.80 |
| 3S:nmole APS bound | | 1.27 | 1.30 | 1.25 | 1.26 | 1.23 | 1.25 | 1.27 |
| AP/prev | | 2.1 | 2.1 | 2.0 | 2.0 | 1.9 | 2.0 | 2.0 |
| AP/PRE cum. | | 10.8 | 11.1 | 11.2 | 10.9 | 10.5 | 11.0 | 10.8 |
| 4M:nmole APM added | | | 3.00 | 3.00 | 3.00 | 3.00 | 3.00 | 3.00 |
| 4M:nmole APM bound | | | 2.37 | 2.38 | 2.36 | 2.40 | 2.31 | 2.37 |
| AP/prev | | | 1.8 | 1.9 | 1.9 | 1.9 | 1.8 | 1.9 |
| AP/PRE cum. | | | 24.8 | 25.3 | 24.5 | 23.9 | 24.6 | 24.3 |
| 5S:nmole APS added | | | | 4.50 | 4.50 | 4.50 | 4.50 | 4.50 |
| 5S:nmole APS bound | | | | 3.62 | 3.65 | 3.66 | 3.57 | 3.63 |
| AP/prev | | | | 1.5 | 1.5 | 1.5 | 1.5 | 1.5 |
| AP/RPE cum. | | | | 46.9 | 45.4 | 44.4 | 45.5 | 44.9 |
| 6M:nmole APM added | | | | | | | | 8.00 |
| 6M:nmole APM bound | | | | | | | | 5.15 |
| AP/prev | | | | | | | | 1.4 |
| AP/RPE total | 3.5 | 10.8 | 24.8 | 46.9 | 45.4 | 44.4 | 45.5 | 74.1 |
| Ab activation | AbS | AbM | AbS | AbM | AbM | AbM | AbM | AbS |
| nmole Ab added | 1.00 | 1.00 | 1.00 | 1.00 | 0.40 | 3.00 | 10.00 | 1.00 |
| nmole Ab bound | 0.97 | 1.00 | 0.99 | 0.98 | 0.39 | 2.85 | 4.41 | 1.00 |
| Ab/RPE | 5.5 | 5.8 | 5.7 | 5.8 | 2.2 | 16.0 | 25.8 | 5.7 |
| MW, kDa | 1551 | 2625 | 4566 | 7670 | 6932 | 8866 | 10476 | 11470 |
| A565 product | 0.1050 | 0.0946 | 0.0848 | 0.0740 | 0.0794 | 0.0762 | 0.0703 | 0.0690 |
| nM RPE product | 53.6 | 48.3 | 43.3 | 37.8 | 40.5 | 38.9 | 35.8 | 35.2 |
| recovery % | 76.0 | 70.2 | 62.4 | 56.1 | 58.2 | 54.5 | 52.5 | 50.0 |
| μg/mL calc | 83.1 | 126.7 | 197.7 | 289.7 | 280.9 | 344.5 | 375.5 | 403.8 |
| μg/mL BCA | 76.8 | 117.2 | 175.3 | 251.1 | 255.7 | 294.4 | 316.6 | 376.8 |
| Activity % | 50.5 | 51.9 | 46.5 | 56.3 | 62.2 | 50.7 | 50.9 | 45.3 |

Results

In the first step (1S), 0.200 nmole SATA-activated RPE was added. The pH in this step was lower than in subsequent steps to slow the reaction, allowing the RPE to disperse throughout the support before linking. A large distance between immobilized molecules of this core protein was desired to minimize the joining of individual conjugates and provide a homogeneous product. Of the 0.200 nmole added SATA-activated RPE, 0.168 to 0.178 bound to the EMCH-activated support.

After deactivating the remaining maleimide groups with MESNA and washing to remove remaining MESNA and activated RPE, 0.8 nmole of maleimide-activated alkaline phosphatase was added (step 2M). This and subsequent layers of activated AP were added under conditions intended to maximize the quantity linking to the growing conjugate. These conditions were excess activated protein, pH 8.0 (to favor the thiol-maleimide reaction), and presence of magnesium ion (to overcome ionic repulsion of the negatively charged proteins). In this example, it was desirable to saturate the growing conjugate with each new layer of protein because this minimizes variation in size between conjugates. In this step (2M), an average of 3.6 molecules of AP were bound for each molecule of RPE present.

In the next step (3S), an average of 2.0 molecules of AP bound for each molecule of AP in the previous layer, giving a total of nearly 11 molecules of AP bound for each molecule of RPE. In step 4M, the uptake of AP again nearly doubled. The one reaction remaining for step 5S showed uptake of only 1.4 molecules of AP per molecule taken up in step 4M, even though a larger excess of activated protein was used.

The IgG used in the final protein addition was activated to either the SH or maleimide derivative to accommodate the activation of the previous AP layer. Since the quantity being added was significantly below the expected saturation level for most of the conjugates there was a risk of uneven uptake of the protein on the conjugates, favoring those located near the surface of the agarose beads over those deeper inside. To promote dispersal of the activated IgG throughout the beads before reaction, the pH for this step was decreased to 7.5, and the magnesium chloride was withheld for 5 minutes after addition of the IgG. Uptake of activated IgG was nearly quantitative for all the reactions except for reaction G, in which excess was used to saturate the conjugate with IgG. In reaction G 1.2 molecules of IgG were absorbed for each molecule of AP in the previous layer, continuing the trend seen with increasing layers of AP.

At the conclusion of incubation of the antibody addition step for each reaction, N-ethylmaleimide was added to deactivate the remaining SH groups on the conjugates. Immediately before the release step, MESNA was added to deactivate remaining maleimide groups. Deactivation of at least one of the activating groups prevents further linkage (dimerization or multimerization) of the conjugates after their release from the support.

Release was effected by treatment with a dilute solution of hydroxylamine at room temperature, and was complete in 1 hour. Hydroxylamine reacts with the hydrazone linkage holding the protein assembly to the support, releasing the conjugate in its final form.

The single hydrazide group remaining after release of the Molecular Conjugate from the agarose may be selectively linked to other compounds. Suitable compounds for linkage through this hydrazide include, but are not limited to, aldehydes, ketones, and activated carboxylic acids.

Reported yields of released conjugate are based on the recovery of bound RPE, measured by its absorbance at 565 nm. These range from 76% for conjugate A, with only a single layer of AP, to 50% for conjugate H, with five layers. The remainder is clearly visible as pink color remaining in the support.

Very little additional conjugate is released from the support after prolonged exposure to hydroxylamine.

The average molecular weight for each of the conjugates was calculated from the uptake of the activated proteins, assuming a single RPE core in each discrete unit of conjugate. This ranges from about 1.5 MDa for Conjugate A to about 11 MDa for Conjugate H. HPLC of the conjugates on a size exclusion column showed peaks consistent with the calculated molecular weights. Monitoring at 565 m showed a decrease in peak intensity with larger conjugate size, reflecting the decreased yields, while monitoring at 280 nm showed an increase in peak intensity, resulting from the higher levels of AP on the larger conjugates.

The RPE present in the conjugate provides a direct measurement of the molar concentration of the conjugates. Its use as the core is intended to assure that one and only one RPE chromophore is present in each final unit of conjugate. Its strong light absorbency (extinction coefficient at 565 nm of 1960000) allows accurate measurement of conjugate concentrations, at least down to the nM range as demonstrated in the present example.

ELISA assays for TSH using the conjugates were performed. When the molar concentration is held constant at 40 pM, and the anti-TSH Antibody is constant at about 6 per conjugate unit, the Vmax for the hydrolysis of PNPP increases in proportion to the amount of Alkaline Phosphatase per Macromolecular Conjugate at each TSH level. Because the molecular weights of the conjugates vary over nearly an order of magnitude, the same experiment was performed with a constant concentration of 200 ng/mL conjugate. In the weight based experiment, conjugate A is at about 3-fold its concentration and conjugate H is at less than ½ its concentration compared with the previous molarity-based experiment. Most of the conjugates give signals similar to those seen with molarity-based concentrations, demonstrating that the signal substantially depends on the alkaline phosphatase content of each conjugate unit not on the number of Macromolecular Conjugates present. Conjugate H did show significantly less signal in the weight-based experiment. Most likely the high molecular weight and low molar concentration result in slow diffusion of the conjugate to the surface containing the bound TSH. Thus, these results demonstrate that the Macromolecular Conjugate of the present invention is superior to that obtained by prior art methods.

The effect of varying antibody content of the conjugate while holding the alkaline phosphatase constant was measured. At equilibrium, with antibody content ranging from 2.2 to 26 per conjugate unit, the signal varies only by about 20%, pointing to the AP content of each conjugate unit as the primary determinant of its ability to generate signal. The rate of diffusion of the conjugate and the rate at which it binds to immobilized analyte can be expected to show dependencies on both the size and the content of binding sites.

### Example 2

In this example, an embodiment of the inventive method is used to prepare a conjugate for a chemiluminescence assay. In the prior art, the chemiluminescent molecule acridinium is often linked directly to an amine group of an antibody of interest. Since the chemiluminescent signal is proportional to the number of acridinium moieties bound per antibody, there is a desire to link as many as possible. However, an excessive amount of acridinium bound to the antibody can interfere with its specific binding to the target molecule, and also can contribute to nonspecific binding to other components of an assay, thus degrading performance.

In this example R-phycoerythrin is heavily substituted with thiol groups, then bound to a solid support. Antibody is activated to contain maleimide groups, and linked to the R-phycoerythrin core. In one portion of this mixture the remaining maleimide groups on the antibody are capped with sulfonate groups using MESNA, while in another portion they are treated with dithiothreitol, which in this case acts as a homobifunctional reagent yielding a thiol in place of the maleimide. After washing excess reagents from the support EMCH is added. The maleimide group on this reagent forms a thioether with the thiol groups on the bound conjugate, giving a conjugate now with hydrazide groups. Both portions are treated with hydroxylamine to release the conjugates, which are dialysed into physiologic buffered saline. The conjugates are now treated with acridinium active ester which, at neutral pH reacts preferentially with the hydrazide groups, placing acridinium at these positions. In the first portion the acridinium is linked primarily on the R-phycoerythrin portion of the conjugate and in the second portion the acridinium is linked on both the R-phycoerythrin and antibody portions.

AbM: To 500 L of antibody at 2.69 mg/mL in physiologic buffered saline was added 100 uL 100 mM triethanolamine at pH 7.7 and 10 uL 100 mM GMBS in DMF. After 50 minutes at room temperature, the mixture was desalted into physiologic buffered saline containing 5 mM EDTA, collecting 1.2 mL.

RS: To 200 uL R-phycoerythrin at 10 mg/mL in 100 mM triethanolamine at pH 7.7 was added 10 μL 500 mM EDTA and 40 μL 100 mM 2-iminothiolane in water. After 55 minutes at room temperature, the mixture was desalted into physiologic buffered saline containing 5 mM EDTA collecting 1.2 mL.

Conjugation of Antibody to R-phycoerythrin

2.0 mL oxidized agarose slurry in a column fitted with a frit was washed with 10 mM sodium phosphate, 150 mM sodium chloride, 0.2% CHAPS, 5 mM EDTA at pH 7.2 (PCE). The column was capped and 500 μL PCE and 50 μL 100 mM EMCH in DMF were added, the mixture vortexed and left at room temperature for 70 minutes. The column was then drained and washed with 2 mL PCE and 6 mL TC8E (100 mM Tris, 0.2% CHAPS, 5 mM EDTA pH 8.0) and placed in an ice bath. To this was added 566 μL (i.e., 4.0 nmole) RS, the mixture vortexed and placed in ice. After 15 minutes with occasional vortexing 100 μL 100 mM MESNA was added. The mixture was vortexed and placed in ice. After 5 minutes the column was drained and washed with 10 mL TC8E. The column was capped and 1.0 mL (i.e., 9.4 nmole) AbM was added. The mixture was vortexed and placed on ice. After 15 minutes with occasional vortexing, the column was drained and washed with 5.5 mL cold TC8E.

The active groups were then capped and the conjugate released as follows. The mixture was divided to two equal portions, labeled A and B, in columns fitted with frits. To A was added 300 μL TC8E and 20 μL 100 mM MESNA to cap maleimide groups on the antibody. To B was added 300 μL TC8E and 20 μL 100 mM DTT to convert antibody maleimide activation with thiol activation. Both A and B were vortexed and placed on ice 10 minutes, then washed with 4.0 mL cold TC8E. The columns were capped, 300 μL TC8E and 20 μL 100 mM EMCH in DMF were added to each, the mixtures vortexed and placed on ice. After 10 minutes the columns were drained and the support washed with 1 mL TC8E and 2 mL TC7E (100 mM Tris, 0.2% CHAPS, 5 mM EDTA pH 7.0). The columns were capped, 300 μL TC7E and 60 μL 50% hydroxylamine added and the mixtures vortexed. After 60 minutes incubating at room temperature, the columns were drained and washed with 2 mL TC7E. The collected effluents were separately dialysed against physiologic buffered saline and labeled 1/15A and B. HPLC of the product on a gel permeation column was consistent with a conjugate of 2 antibodies on a R-phycoerythrin core.

The conjugates were then reacted with acridinium active ester as follows. To 700 μL of A and B in separate tubes was added 20 μL of acridinium active ester at 5 mg/mL in DMF. After 4.5 hours at room temperature, the mixtures were passed through desalting columns equilibrated with physiologic buffered saline, collecting 1.0 mL each, labeled 1/16A and B.

The absorbances of the conjugates were measured at 280 nm, 370 nm and 565 nm. For calculations, ext565=1960000 for R-phycoerythrin chromophore (RPE) and ext370=14650 for acridinium chromophore (Ac). The ratio A₃₇₀/A₅₆₅=0.118 was calculated from 1/15A and B. This was used to calculate the A₃₇₀ contributed by R-phycoerythrin for 1/16A and B. This was subtracted from the measured A₃₇₀ to get A₃₇₀ contributed by acridinium for 1/16A and B. From this was calculated the concentration of acridinium in μM units and the Substitution Ratio (SR).

| | A₂₈₀ | A₃₇₀ | A₅₆₅ | μM protein | A₃₇₀ RPE | A₃₇₀ Ac | μM Ac | SR |
|---|---|---|---|---|---|---|---|---|
| 1/15A | 0.4628 | 0.1273 | 1.0855 | 0.554 | | | | |
| 1/15B | 0.4269 | 0.1180 | 0.9882 | 0.504 | | | | |
| 1/16A | 0.3126 | 0.2388 | 0.5992 | 0.306 | 0.071 | 0.168 | 11.5 | 37.5 |
| 1/16B | 0.2979 | 0.2158 | 0.4661 | 0.238 | 0.055 | 0.161 | 11.0 | 46.2 |

1/16A shows SR (Acridinium/Conjugate) very close to the number of SH groups per RPE measured after its original activation. 1/16B shows more, presumably from the additional active groups used on the antibody.

While the invention has been described in detail and with reference to specific embodiments, it will be apparent to one skilled in the art that various changes and modifications may be made to such embodiments without departing from the spirit and scope of the invention.

What is claimed is:

1. A method of making a suspended or soluble protein conjugate, the method comprising the steps of:
a) providing a reactive surface, protein that is capable of forming a stable, disruptable bond with the reactive solid-surface, and a Second Macromolecule,
b) contacting the protein to the reactive solid-surface to form a surface-bound protein complex,
c) if necessary, activating the protein, or Second Macromolecule, or both,
d) contacting the surface bound protein with a reactive Second Macromolecule to form a solid-surface:protein: Second Macromolecule stable complex,
e) disrupting the stable bond between the solid-surface and the protein conjugate in a liquid medium to yield a suspended or soluble protein conjugate comprising the protein and the Second Macromolecule.

2. A method of making a suspended or soluble Macromolecular Conjugate, the method comprising the steps of:
a) providing a reactive surface,
b) providing a First Macromolecule that is capable of forming a stable, disruptable bond with the reactive solid-surface,
c) contacting the First Macromolecule to the reactive solid-surface to form a surface-bound First Macromolecule complex,
d) if necessary, activating the First Macromolecule, or Second Macromolecule, or both,
e) contacting the surface bound First Macromolecule with a reactive Second Macromolecule to form a solid-surface:First Macromolecule:Second Macromolecule stable complex,
f) disrupting the stable bond between the solid-surface and the First Macromolecule conjugate in a liquid medium to yield a suspended or soluble Macromolecular Conjugate conjugate comprising the First Macromolecule and the Second Macromolecule,
wherein the First Macromolecule comprises a plurality of reactive moieties capable of interacting with the Second Macromolecule, and the Second Macromolecule comprises a plurality of reactive moieties capable of interacting with the First Macromolecule.

3. The method of claim 2, wherein the First Macromolecule is bound to the solid-surface through a disruptable covalent bond.

4. The method of claim 2, wherein Second Macromolecule is attached to the First Macromolecule through a bifunctional linker.

5. The method of claim 4, wherein the bifunctional linker is selected from the group consisting of N-Succinimidyl S-Acetylthiopropionate, N-Succinimidyl S-Acetylthioacetate, 2-Iminothiolane (Trauts reagent), 4-Succinimidyloxycarbonyl-Methyl-(2-Pyridyldithio)-Toluene Sulfosuccinimidyl, 4-[N-maleimidomethyl]-cyclohexane-1-carboxylate, N-[gamma-Maleimidobutyryloxy]sulfo-succinimide ester, N-(K-Maleimidoundecanoyloxy) Sulfosuccinimide Ester, Maleimidoacetic Acid N-Hydroxysuccininide Ester, N-(Epsilon-Maleimidocaproic Acid) Hydrazide, N-(K-Maleimidoundecanoic Acid) Hydrazide, N-(Beta-Maleimidopropionic Acid) Hydrazide, and 3-(2-Pyridyldithio)Propionyl Hydrazide.

6. The method of claim 5, wherein the First Macromolecule is a protein, and the protein comprises reactive sulfhydryl moieties, and the solid-surface comprises maleimide moieties bound to the surface via hydrazone linkage.

7. The method of claim 4 further comprising:
rendering the solid-surface unreactive with the First Macromolecule after completing step (c).

8. The method of claim 7, further comprising
contacting the solid surface-bound: First Macromolecule: Second Macromolecule with a Third Macromolecule that is reactive with the Second Macromolecule to form a surface-bound complex comprising at least one unit of the Third Macromolecule linked to the First Macromolecule through the Second Macromolecule, wherein
the First Macromolecule and the Third Macromolecule may be the same or different.

9. The method of claim 8, further comprising
after contacting the Second Macromolecule with the Third Macromolecule: adding a deactivating reagent, or placing the surface-bound complex under conditions, sufficient to render any non-reacted reactive moieties on the Second Macromolecule in the complex non-reactive to the reactive moieties used on the Third Macromolecule,
removing any excess deactivating reagent, or altering the conditions, such that the reactive moieties on additional macromolecules added to the reaction would not be deactivated, and contacting the surface-bound complex with a Fourth Macromolecule under conditions sufficient to form a bond between the Third Macromolecule and the Fourth Macromolecule, wherein
the Second Macromolecule and the Fourth Macromolecule may be the same or different.

10. The method of claim 8, further comprising one or more sequential additions of macromolecules.

11. The method of claim 2, wherein the solid surface is selected from the group consisting of agarose, polyacrylamide, and polystyrene, the reactive moiety on the solid support is a maleimide linked to the support via a hydrazone group, the First Macromolecule is linked to the maleimide on the solid support via the thioether group formed by reaction of the sulfhydryl group with the maleimide group.

12. The method of claim 2, wherein the stable bond between the solid-surface and the First Macromolecule conjugate is disrupted by contacting the bond with a reagent specific to the disruptable bond, thereby releasing the conjugate from the solid surface.

13. The method of claim 12, wherein the disruption of the bond yields a hydrazide on the First Macromolecule of the Macromolecular Conjugate, the method further comprising reacting the hydrazide with a compound to change the character of the conjugate.

14. The method of claim 2, wherein a residual reactive moiety on the First Macromolecule or a residual reactive moiety on the Second Macromolecule that does not react to bond the First Macromolecule with the Second Macromolecule is deactivated with a capping compound.

15. The method of claim 15, wherein the capping compound is zwitterionic.

16. The method of claim 15, wherein the capping compound is a polymer selected from the group consisting of dextran, polyethylene glycol, and polysaccharides.

17. The method of claim 15, wherein the capping compound is polymer selected from the group consisting of a polypeptide and a nucleic acid.

18. The method of claim 2, wherein the First Macromolecule is provided in a composition comprising a second molecule that reacts with the solid surface and does not react with the Second Macromolecule.

19. A conjugate produced by the method of claim 1, wherein the conjugate comprises only one antibody.

20. A conjugate produced by the method of claim 1, wherein the conjugate comprises a predetermined number of antibodies, wherein the number of antibodies is between 2 and 30.

21. A composition comprising a population of conjugates, wherein each conjugate of the population comprises a single antibody.

22. A composition comprising a population of conjugates, wherein each conjugate of the population comprises a predetermined number of antibodies and the number of antibodies is between 2 and 30.

23. A conjugate comprising three layers of macromolecules, wherein at least two of the three layers comprise a plurality of macromolecules, and wherein the layers form a surface in three dimensions.

24. A conjugate comprising
a specific binding member,
a plurality of end-point molecules, and
a plurality of spacer molecules, wherein
the spacer molecules substantially separate the end-point molecules.

25. A population of conjugates, wherein
substantially each conjugate of the molecule comprises from 1 to 30 molecules of a specific binding members,
each of the specific binding members is disposed on the surface of the conjugate, and substantially each conjugate comprises a core, wherein the core comprises at least one molecule that is not disposed on the surface of the conjugate.

26. A conjugate prepared according to claim 1 in which one of the macromolecules, preferably the first, comprises a chromophore rendering it optically distinguishable from the other macromolecules of the conjugate such that the final conjugate can be quantified according to the optical absorbance or fluorescence of said chromophore.

27. A conjugate according to claim 26, wherein the macromolecule is selected from the list R-phycoerythrin, B-phycoerythrin, and allophycocyanin.

28. A kit comprising a reactive conjugate complex, and a cleavage reagent wherein,
the reactive conjugate complex comprises a solid bonded with a First Macromolecule covalently complexed with a Reactive Macromolecule, and
the cleavage reagent is capable of cleaving the bond between the solid and the First Macromolecule.

29. The kit of claim 28, further comprising an activation reagent, wherein the activation reagent can be contacted to a protein of interest to make it reactive with the Reactive Macromolecule.
